(19) United States
(12) Patent Application Publication
(10) Pub. No.: US 2005/0124031 A1
(43) Pub. Date: Jun. 9, 2005

(54) Biosyntheticse genes of blakeslea trispora beta-carotene that code for lycopene cyclase/phytoene synthase (carrp) and phytoene dehydrogenase (carb)

(75) Inventors: Marta Rodriguez Saiz, Leon (ES); Ana Teresa Marcos Rodriguez, Leon (ES); Bruno Diez Garcia, Leon (ES); Juan Luis Dela Fuente Moreno, Leon (ES); Jose Luis Barredo Fuente, Leon (ES)

Correspondence Address: LADAS & PARRY, 26 WEST 61ST STREET, NEW YORK, NY, 10023, US

(73) Assignee: VITATENE, S.A., Leon (ES)

(21) Appl. No.: 10/489,895

(22) PCT Filed: Sep. 26, 2002
(86) PCT No.: PCT/ES02/00452

(30) Foreign Application Priority Data
Sep. 26, 2001 (ES) P200102161

(51) Int. Cl. C07H 21/04; C12P 23/00; C12N 9/04; C12N 15/74

(52) U.S. Cl. 435/67; 435/190; 435/252.31; 435/320.1; 536/23.2

(57) ABSTRACT

Biosynthetic genes of β-carotene from Blakeslea trispora that code for lycopene cyclase/phytoene synthase (carRP) and phytoene dehydrogenase (carB). The carRP gene, characterized by the DNA sequence SEQ ID NO:1, codes for the amino acid sequence SEQ ID NO:3, which has the lycopene cyclase/phytoene synthase enzymes. For its part, the carB gene, characterized by the DNA sequence SEQ ID NO:2, codes for the amino acid sequence SEQ ID NO:4, which possesses phytoene dehydrogenase enzyme activity. In addition, (i) procedures for constructing plasmids for the expression of additional copies of the aforesaid genes and (ii) methods for the expression of heterologous genes under the control of the promoters of said genes, are described.

### FIELD OF THE INVENTION

The present invention relates to two new genes of Blakeslea trispora designated carRP and carB. The gene carRP codes for the bifunctional enzyme lycopene cyclase/phytoene synthase, whereas carB codes for the enzyme phytoene dehydrogenase. Both genes are involved in the biosynthetic pathway of β-carotene of B. trispora (see scheme).

### STATE OF THE ART

The carotenoids are pigments of an isoprenoid nature that are synthesized by certain bacteria, fungi and photosynthetic organisms. Owing to their beneficial effects on health and their attractive colors, the carotenoids are of considerable commercial importance as coloring matter and food additives. β-Carotene is a carotenoid whose chemical synthesis has been known since 1956. It has a molecular weight of 536.9, and a molecule (C₄₀H₅₆) with eleven conjugated double bonds. Its color is reddish-violet in the crystalline state, yellowish-orange in oily solution, and orange in aqueous dispersion. Synthetic β-carotene possesses the all-trans isomeric configuration, whereas the β-carotene obtained from various natural sources has various forms: mono-cis, di-cis and poly-cis.

The production of carotenoids by microbial biosynthesis is a classic example of competition between chemical and biological processes. Biotechnological processes show, among other things, the advantage of making it possible to obtain, in a simple way, the carotenoids of more complex structure, such as the conformational isomers that only exist in natural form. The industrial biotechnological processes for the production of β-carotene, competing with chemical synthesis, are based on the use of the alga Dunaliella salina and the fungus B. trispora. The production process with B. trispora involves carrying out a mixed fermentation of the (+) and (−) strains for achieving maximum production of β-carotene. This leads to the biosynthesis of trisporic acids, which induce the production of β-carotene. β-Carotene is produced both by the (+) strain and by the (−) strain, being metabolized by both to retinal and then to 4-dihydrotrisporol. The (+) strain uses 4-dihydrotrisporol as substrate for forming dihydrotrisporic acid and its methyl ester (methyl-4-dihydrotrisporate). For its part, the (−) strain metabolizes 4-dihydrotrisporol to trisporol. Finally, the methyl-4-dihydrotrisporate is converted to trisporic acid by the (−) strain and the trisporol is converted to trisporic acid by the (+) strain. This description of the biosynthesis of the trisporic acids is a simplification, since the process generates many co-metabolites, some of which are common to both strains (+) and (−), but others are specific to one of them. The relative amounts of these co-metabolites vary depending on the strains.

Despite the great biotechnological importance of the fungus B. trispora, scientific knowledge about the biosynthesis of β-carotene is scant, since: (i) basic procedures have not yet been described for its genetic manipulation, i.e. cloning of biosynthetic/regulatory genes and transformation and (ii) there is a lack of information on the characterization of enzymes involved in the biosynthesis of β-carotene. Nevertheless, the biosynthetic pathway of β-carotene (see scheme) has been described in phylogenetically related fungi such as Phycomyces blakesleeanus and Mucor circinelloides (Arrach N. et al. (2001) Proceedings of the National Academy of Sciences USA 98: 1687-1692; Velayos A. et al. (2000) European Journal of Biochemistry 267: 5509-5519). At least three enzymes are necessary for this biosynthesis: (i) phytoene synthase, which joins together two molecules of geranylgeranyl pyrophosphate generating phytoene, (ii) phytoene dehydrogenase, which introduces four double bonds in the phytoene molecule to synthesize lycopene, and (iii) lycopene cyclase, which, using lycopene as substrate, takes on the task of forming the rings located at both ends of the β-carotene molecule.

Analysis of mutants of B. trispora has led to the conclusion that the biosynthetic pathway of β-carotene in this fungus is similar to that described for P. blakesleeanus (Metha B. J. and Cerdá-Olmedo E. (1995) Applied Microbiology and Biotechnology 42: 836-838). In the case of P. blakesleeanus, the yellow color of its mycelium can be altered by mutation, giving rise to strains with mycelium colored red, white or various shades of yellow. The red mutants accumulate lycopene, whereas the white ones lack production of carotenoids or accumulate phytoene. Using complementation analysis, two genes have been identified called carB and carRA, whose products are probably organized in an enzyme complex in which the four dehydrogenations are catalyzed by four identical units of phytoene dehydrogenase and the two cyclizations are catalyzed by two identical units of lycopene cyclase (Candau R. et al. (1991) Proceedings of the National Academy of Sciences USA 88: 4936-4940). Both the carB gene (Ruiz-Hidalgo M. J. et al. (1997) Molecular and General Genetics 253: 734-744) and the carRA gene (Arrach N. et al. (2001) Proceedings of the National Academy of Sciences USA 98: 1687-1692) have been cloned and characterized. The carRA gene has two different domains: (i) the domain R, which is located nearer to the 5′ end and codes for the lycopene cyclase activity and (ii) the domain A, which is responsible for the phytoene synthase activity. Moreover, the carB gene of P. blakesleeanus has been expressed in M. circinelloides (Ruiz-Hidalgo M. J. et al. (1999) Current Microbiology 39: 259-264).

The genes carB (Velayos A. et al. (2000) Planta 210: 938-946) and carRP (Velayos A. et al. (2000) European Journal of Biochemistry 267: 5509-5519) of M. circinelloides have also been cloned and characterized. The carRP gene codes for a bifunctional enzyme lycopene cyclase/phytoene synthase with two domains: (i) the domain R, which is situated nearer the 5′ end and codes for the lycopene cyclase activity and (ii) the domain P, located at the 3′ end and responsible for the phytoene synthase activity. The domain R is functional even in the absence of the domain P, whereas the domain P requires the presence of the domain R for proper functioning.

### DETAILED DESCRIPTION OF THE INVENTION

The present invention describes for the first time the carB and carRP genes of B. trispora. As has been described previously for P. blakesleeanus and M. circinelloides, the carB gene codes for phytoene dehydrogenase and the carRP gene codes for a bifunctional enzyme lycopene cyclase/phytoene synthase with a domain R (nearer the 5′ end and coding for the lycopene cyclase activity) and a domain P (located at the 3′ end and responsible for the phytoene synthase activity). Concretely, the product encoded by carB carries out the conversion of phytoene to lycopene and the bifunctional enzyme encoded by carRP catalyzes both the biosynthesis of phytoene from geranylgeranyl-PP and the conversion of lycopene to β-carotene. Both genes: (i) are involved in the biosynthetic pathway of β-carotene, (ii) are contiguous in the genome and (iii) are expressed under the control of a bidirectional promoter located between the two. This bidirectional promoter permits gene expression in one direction or in the opposite direction. Mutants lacking either of these two genes are not capable of producing β-carotene, but accumulate the corresponding biosynthetic intermediates (see scheme).

B. trispora is a fungus that is of considerable industrial importance for the biotechnological production of β-carotene. In fact, this process is becoming competitive with the synthetic process used industrially at present. The aforementioned genes carRP and carB can be used, for example, for (i) improving the yield of β-carotene by increasing its expression and (ii) modifying the biosynthetic pathway of β-carotene by generating strains capable of biosynthesis of other carotenoids. Gene expression of carRP and carB can be increased by inserting extra copies of said genes in B. trispora, either in their natural state or expressed under the control of strong promoters. Modification of the biosynthetic pathway of β-carotene can be achieved, for example, by inactivating the lycopene cyclase activity by manipulation of the carRP gene. In this way, strains would be generated that are able to produce lycopene, a carotenoid of considerable industrial and commercial interest.

The scheme on the next page shows the genes and enzymes involved in the biosynthetic pathway of β-carotene of B. trispora. The phytoene synthase activity joins together two molecules of geranylgeranyl pyrophosphate, generating phytoene. The phytoene dehydrogenase activity introduces four double bonds in the phytoene molecule to synthesize lycopene. The lycopene cyclase activity, using lycopene as substrate, forms the rings located at both ends of the β-carotene molecule.

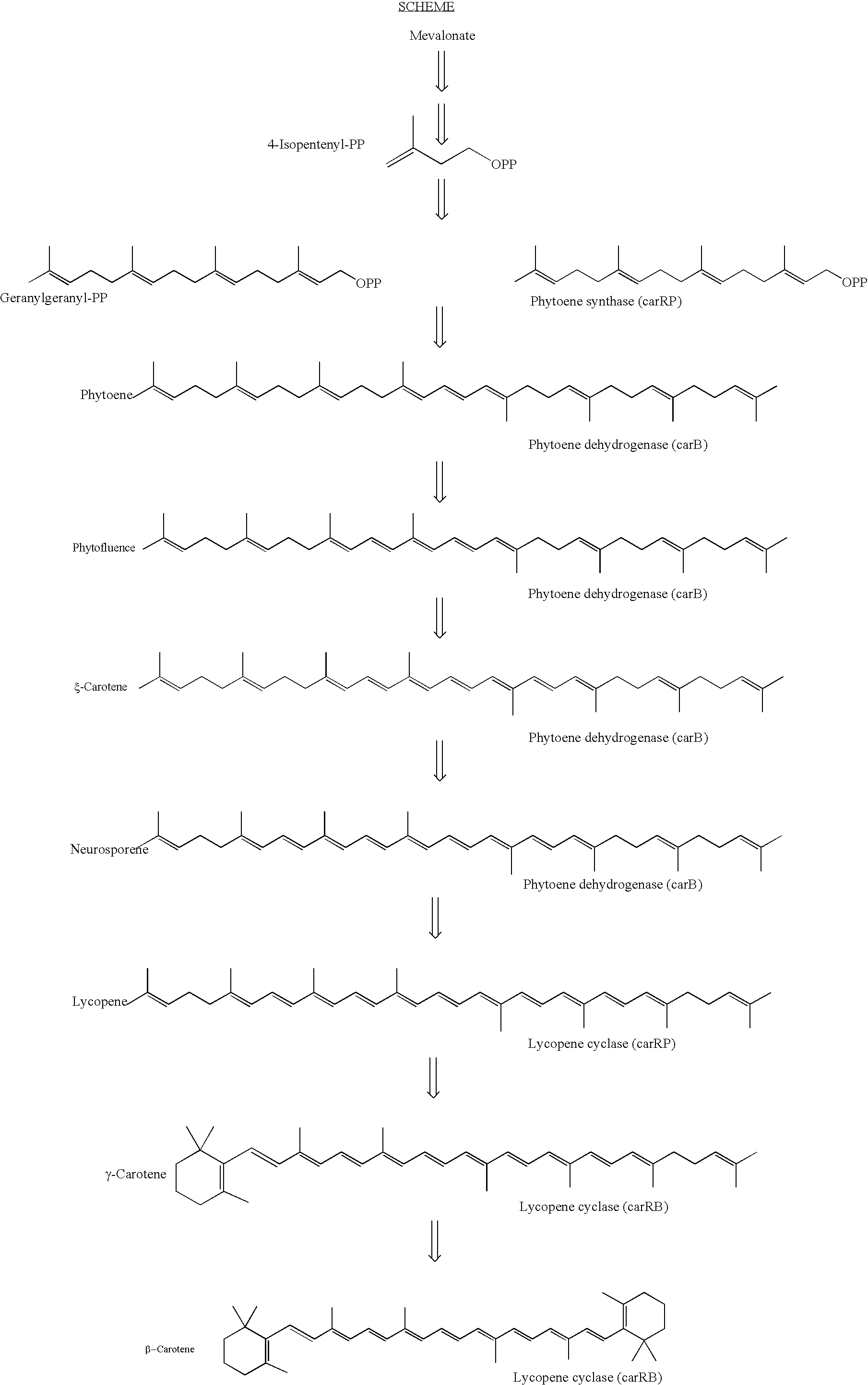

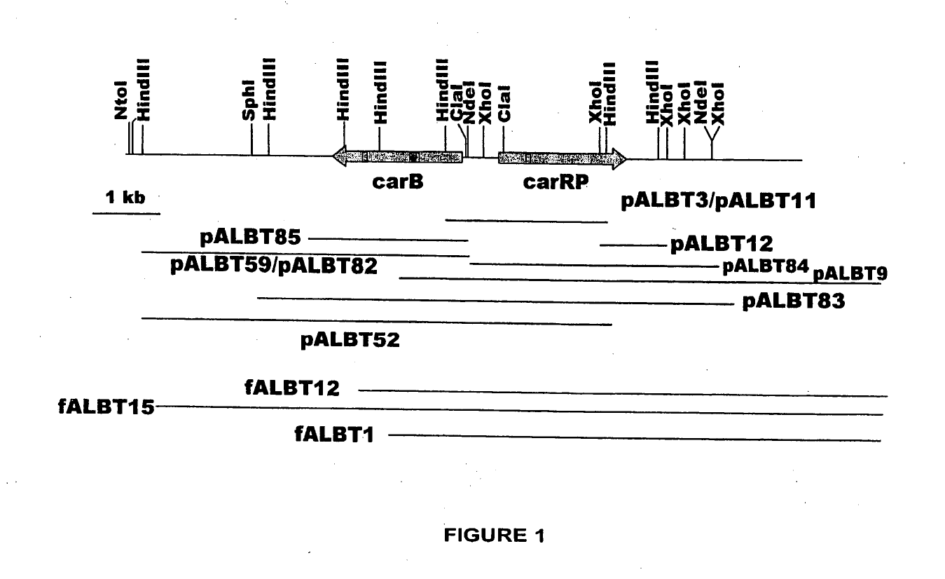

The genomic DNA of the fungus B. trispora was used for constructing a gene library in the phage vector λ-GEM12. For this, partial digestion was effected with the restriction endonuclease Sau3AI and the resulting fragments were joined to λ-GEM12, as described in Sambrook J. et al. (1989) Molecular Cloning: A Laboratory Manual, Cold Spring Harbor Laboratory, Cold Spring Harbor, N.Y., USA. The genes carRP (lycopene cyclase/phytoene synthase) and carB (phytoene dehydrogenase) were cloned by screening the said gene library of B. trispora using a probe corresponding to the carRP gene of Mucor circinelloides. The probe was obtained by PCR (polymerase chain reaction) using primers designed on the basis of the sequence of the carRP gene of Mucor circinelloides (Velayos A. et al. (2000) European Journal of Biochemistry 267: 5509-5519). In this way a 560-bp DNA fragment was amplified and was used for scanning the gene library, isolating 4 recombinant phages designated fALBT1, fALBT4, fALBT12 and fALBT15 (FIG. 1). Said clones were analyzed with a series of restriction endonucleases and then a 2.4-kb HindIII fragment was subcloned in plasmid vectors of E. coli. The restriction map of the aforesaid fragment is shown in the Scheme. Next, the sequence of the 2430 bp included in the aforementioned HindIII fragment was determined, and two incomplete open reading frames (ORFs) were found, which were transcribed in opposite directions. On the basis of their similarity with sequences present in databases they were designated carRP (SEQ ID NO:1) and carB (SEQ ID NO:2). Subsequent subcloning and sequencing of the DNA regions adjacent to the ends of the aforesaid 2.4 kb fragment HindIII made it possible to complete the nucleotide sequence of both ORFs.

The ORF corresponding to the carRP gene has a length of 1894 bp and is interrupted by a 70-bp intron located between positions 406 and 475. Said ORF codes for a protein of 608 amino acids, 69,581 Da and an isoelectric point of 7.78. Comparison of its deduced amino acid sequence (SEQ ID NO:3) with the SwissProt database shows great similarity with genes that code for the lycopene cyclase/phytoene synthase enzymes of M. circinelloides (67%), P. blakesleeanus (55%) and Neurospora crassa (29%) (Velayos A. et al. (2000). European Journal of Biochemistry 267: 5509-5519; Arrach N. et al. (2000). Proceedings of the National Academy of Sciences USA 98: 1687-1692; Schmidhauser T. J. et al. (1994). The Journal of Biological Chemistry 269: 12060-12066).

The ORF corresponding to the carB gene has a length of 1955 bp and is interrupted by two introns of 141 bp and 68 bp, located between positions 594-734 and 1584-1651 respectively. This ORF codes for a protein of 582 amino acids, 66,426 Da and an isoelectric point of 6.9. Comparison of its deduced sequence of amino acids (SEQ ID NO:4) with the SwissProt database shows great similarity with genes that code for the phytoene dehydrogenase enzyme in M. circinelloides (80%), P. blakesleeanus (72%) and Neurospora crassa (48%) (Velayos A. et al. (2000). Planta, 210: 938-946; Ruiz-Hidalgo et al. (1997). Molecular and General Genetics 253: 734-744; Ruiz-Hidalgo et al. (1999). Current Microbiology 39: 259-264; Schmidhauser et al. (1990). Molecular and Cellular Biology 10: 5064-5070).

A 6.9-kb DNA fragment that includes both genes (carRP and carB) was subcloned, using suitable restriction targets, in plasmid vectors that can be incorporated in one or more copies in the genome of B. trispora, which permit expression of these genes in the host fungus. Similarly, the promoters of the carRP and carB genes can be obtained (using suitable restriction targets or by PCR) and used for expressing homologous and heterologous genes in B. trispora. The following examples describe the expression of the phleomycin resistance gene (ble^{R}) of Streptoalloteichus hindustanus (Drocourt D. et al. (1990). Nucleic Acids Research 18: 4009) under the control of the promoters of the carRP and carB genes. As was described earlier, the promoters of the carRP and carB genes can be used for the correct expression of heterologous genes in B. trispora or for the overexpression of homologous genes that are weakly transcribed.

The expression in B. trispora of biosynthetic genes of xanthophylls can give rise to transformants capable of biosynthesizing carotenoids such as zeaxanthin, canthaxanthin, astaxanthin or new carotenoids. In addition, by means of gene interruption, it would be possible for the biosynthetic pathway of β-carotene to be blocked, obtaining transformants capable of producing, for example, lycopene as the end product.

Deposit of Microorganisms Under the Budapest Treaty.

Two strains of Escherichia coli bearing the plasmids pALBT9 (which contains the carRP gene) and pALBT52 (which contains the carB gene) were deposited, in accordance with the provisions of the Budapest Treaty, in the Spanish Type Culture Collection (CECT), Department of Microbiology of the Biology Faculty of the University of Valencia, Campus de Burjasot, 46100 Burjasot, Valencia, (Spain), with the numbers CECT 5982 and CECT 5981 respectively on 09.17.01.

The following examples describe the present invention in detail and without limitation.

### EXAMPLE 1

Construction of Gene Libraries of the (+) and (−) Strains of B. trispora.

Gene libraries of the (+) and (−) strains of B. trispora were constructed in the following way: A total of 300 μg of whole DNA was digested partially with 20 units of Sau3AI in a reaction volume of 600 μl at 37° C. and 3 aliquots of 200 μl were collected at 45 seconds, 1 minute and 2 minutes respectively, digestion being stopped with cold EDTA 20 mM. After confirming the digests in 0.7% agarose gel, they were mixed, heated at 68° C. for 10 minutes, left to cool slowly to room temperature and placed on a sucrose gradient (10-40%) of 38 ml. This gradient was centrifuged at 26,000 rpm for 24 hours at 25° C., after which 0.5-ml aliquots were collected, and 10 μl of each one was analyzed in 0.4% agarose gel. The aliquots whose DNA had a size between 18 and 22 kb were mixed and then diluted with distilled water to approximately 10% sucrose. Then the DNA was precipitated with ethanol, resuspended in 50 μl of TE buffer and 3 μl of this last-mentioned solution was analyzed in 0.4% agarose gel. It was confirmed in this gel that the size of the DNA fragments was correct and that their concentration was approximately 50 ng/μl.

In parallel, the DNA of the bacteriophage λ-GEM12 (Promega) was prepared by methods described previously (Sambrook J. et al. (1989) Molecular Cloning: A Laboratory Manual, Cold Spring Harbor Laboratory, Cold Spring Harbor, N.Y., USA). 50 μg of DNA from the bacteriophage was digested with the endonucleases BamHI and EcoRI at 37° C. for 2 hours. The double digest was extracted with phenyl/CIA and CIA, precipitated with ethanol and resuspended in 50 μl of TE buffer. After collecting a 2-μl aliquot, MgCl₂ was added to the rest, to 10 mM, then it was incubated at 42° C. for 1 hour to promote circularization of the arms of the vector on its cohesive ends. A 2-μl fraction was collected again and was analyzed together with the previous one in 0.5% agarose gel to confirm correct circularization on the cohesive ends and determine its approximate concentration (around 100 ng/μl).

Next, a series of ligations was carried out using 0.25 μg of insert and quantities of vector between 0.25 and 0.75 μg, varying the insert/vector ratio. The reactions were incubated at 12-14° C. for 16 hours. Encapsidation of the recombinant phage DNA produced after ligation was performed with ‘Packagene’ (Promega) in vitro packaging extracts. With the resultant from the encapsidation reaction resuspended in 500 μl of SM, infections of E. coli NM538 (Promega) were carried out to determine the number of phages present and of E. coli NM539 (Promega) with the aim of determining the percentage of recombinant phages. E. coli NM539 is a lysogenic strain of the P2 phage and only produces lysis plaques when the phage infecting it lacks the dispensable central region.

The titer of the gene libraries constructed proved to be as follows: 125,000 pfu for B. trispora (+) and 50,000 pfu for B. trispora (−). In both cases around 80% of the phages were carriers of an exogenous DNA fragment.

### EXAMPLE 2

Cloning and Characterization of the carB and carRP Genes of B. trispora.

The two gene libraries were transferred to nitrocellulose filters and were scanned with a 560-bp probe corresponding to the carRP gene of M. circinelloides. This probe was obtained by PCR amplification using the primers #61 (SEQ ID NO:5) and #62 (SEQ ID NO:6), which were designed as a function of the DNA sequence of the carRP gene of M. circinelloides (Velayos A. et al. (2000) European Journal of Biochemistry 267: 5509-5519). The process for selecting positive phages was carried out in accordance with hybridization methods previously described (Sambrook J. et al. (1989) Molecular Cloning: A Laboratory Manual, Cold Spring Harbor Laboratory, Cold Spring Harbor, N.Y., USA). Once the process of prehybridization, hybridization, washing and autoradiography was completed, 4 clones were selected, designated fALBT1, fALBT4, fALBT12 and fALBT15, which produced positive signals with probe DNA. The DNA that includes the carRP and carB genes claimed in this invention can be obtained by digesting the phages fALBT4 or fALBT15 (FIG. 1) with suitable restriction enzymes using standard techniques (Sambrook J. et al. (1989) Molecular Cloning: A Laboratory Manual, Cold Spring Harbor Laboratory, Cold Spring Harbor, N.Y., USA). DNA fragments containing the sequences SEQ ID NO:1 and SEQ ID NO:2 can be obtained with one or more restriction enzymes (FIG. 1) and subcloned in a suitable vector (for example pBluescript).

A 2.4-kb HindIII DNA fragment obtained from the fALBT1 phage (FIG. 1) was subcloned in the HindIII restriction site of the plasmid pBluescript I KS (+) in the two possible orientations. The resulting plasmids were designated pALBT3 and pALBT11. These plasmids were subjected to sequence deletions with the ‘Erase-a-base’ kit (Promega) in accordance with the manufacturer's instructions. The sequence reactions of the deleted clones were carried out with the ‘Dye Terminator Cycle Sequencing Ready Reactions Kit’ and the DNA fragments were separated in an ABI PRISM automatic sequencer (Perkin Elmer) following the manufacturer's instructions. In this way we obtained the sequence of nucleotides, on both chains, of a total of 2430 bp. Using the Geneplot program (DNASTAR), two incomplete ORFs were identified which corresponded to the carRP and carB genes. With the aim of completing the nucleotide sequence of the two genes, we carried out subcloning and subsequent sequencing of the DNA regions adjacent to the aforementioned HindIII fragment. Starting from the fALBT1 phage, a 1.0-kb XhoI fragment was subcloned in pBluescript I KS (+) (FIG. 1), which includes the 3′ end of the carRP gene, giving rise to the pALBT12 plasmid. Sequencing for both chains of the fragment included in said plasmid made it possible to complete the nucleotide sequence of the carRP gene.

In addition, with the aim of determining the sequence of the 3′ region of the carB gene, the subcloning of a 5.1-kb ClaI-NotI fragment obtained from fALBT15 was performed in pBluescript I KS (+). The plasmids obtained (both orientations), designated pALBT59 and pALBT82, were submitted to sequence deletions with the ‘Erase-a-base’ kit (Promega) according to the manufacturer's instructions. The sequence reactions of the deleted clones were carried out with the ‘Dye Terminator Cycle Sequencing Ready Reactions Kit’ and the DNA fragments were separated in an ABI PRISM automatic sequencer (Perkin Elmer) following the manufacturer's instructions.

The complete nucleotide sequence of the carRP gene revealed an ORF of 1894 bp, interrupted by an intron of 70 bp, situated between positions 406475 and flanked by the following intron/exon splicing sequences: 5′ (g/GTATGCA) and 3′ (TTAG/c). It is a question of conserved sequences when they are compared with the consensus sequences described for fungi: 5′ (g/GTAHGTYW) and 3′ (MYAG/g). This gene codes for a polypeptide of 608 amino acids with a deduced molecular weight of 69,581 Da and an isoelectric point of 7.78. The protein deduced from the sequence (SEQ ID NO:3) shows great similarity with the enzymes with lycopene cyclase/phytoene synthase activity of M. circinelloides (67%), P. blakesleeanus (55%) and N. crassa (29%) described in the databases (Velayos A. et al. (2000). European Journal of Biochemistry 267: 5509-5519; Arrach N. et al. (2000). Proceedings of the National Academy of Sciences USA 98: 1687-1692; Schmidhauser T. J. et al. (1994). The Journal of Biological Chemistry 269: 12060-12066).

The carB gene, 1955 bp in length, is interrupted by two introns of 141 and 68 bp, located between positions 594-734 and 1584-1651 respectively, and flanked by conserved fungal intron/exon splicing sequences [5′ (g/GTAAGTA) and 3′ (ATAG/t) for the 141-bp intron and 5′ (g/GTAATAC) and 3′ (GTAG/t) for the 68-bp intron] similar to those described for the carRP gene. The carB gene codes for a polypeptide of 582 amino acids with a deduced molecular weight of 66,426 Da and an isoelectric point of 6.9. The protein deduced from the sequence (SEQ ID NO:4) shows great similarity with the enzymes with phytoene dehydrogenase activity of M. circinelloides (80%), P. blakesleeanus (72%) and N. crassa (48%) described in the databases (Velayos A. et al. (2000). Planta, 210: 938-946; Ruiz-Hidalgo et al. (1997). Molecular and General Genetics 253: 734-744; Ruiz-Hidalgo et al. (1999). Current Microbiology 39: 259-264; Schmidhauser et al. (1990). Molecular and Cellular Biology 10: 5064-5070).

### EXAMPLE 3

Construction of Plasmids for Heterologous Expression in B. trispora.

The pALFleo7 plasmid (Diez B. et al. (1999) Applied Microbiology and Biotechnology 52: 196-207) was used as the starting point for constructing heterologous expression plasmids in B. trispora. Said plasmid contains the phleomycin resistance gene (ble^{R}) of S. hindustanus (Drocourt D. et al. (1990). Nucleic Acids Research 18: 4009) with a restriction site for NcoI located on the ATG translation start codon and a restriction site for BamHI preceding the NcoI. pALFleo7 also possesses the trpC gene terminator of Aspergillus nidulans (TtrpC). The cluster formed by the ble^{R} gene and TtrpC was purified by double digestion of pALfleo7 with BamHI plus BglII and was subcloned at the BamHI site of pBluescript I KS(+). The resulting plasmid was designated pALfleo8. Next, two NcoI sites were inserted in the ATG translation start codons of the carB and carRP genes, by directed mutagenesis using the ‘QuikChange™ Site-Directed Mutagenesis Kit’ (Stratagene). For inserting the NcoI cutting site in the carB gene, the following oligonucleotides were designed, represented by SEQ ID NO:7 and SEQ ID NO:8. For inserting the NcoI cutting site in the carRP gene, the oligonucleotides represented by SEQ ID NO:9 and SEQ ID NO:10 were designed. In both cases pALBT3 was used as template for PCR amplification. Once the corresponding mutations were inserted, the pALBT56 plasmid was obtained.

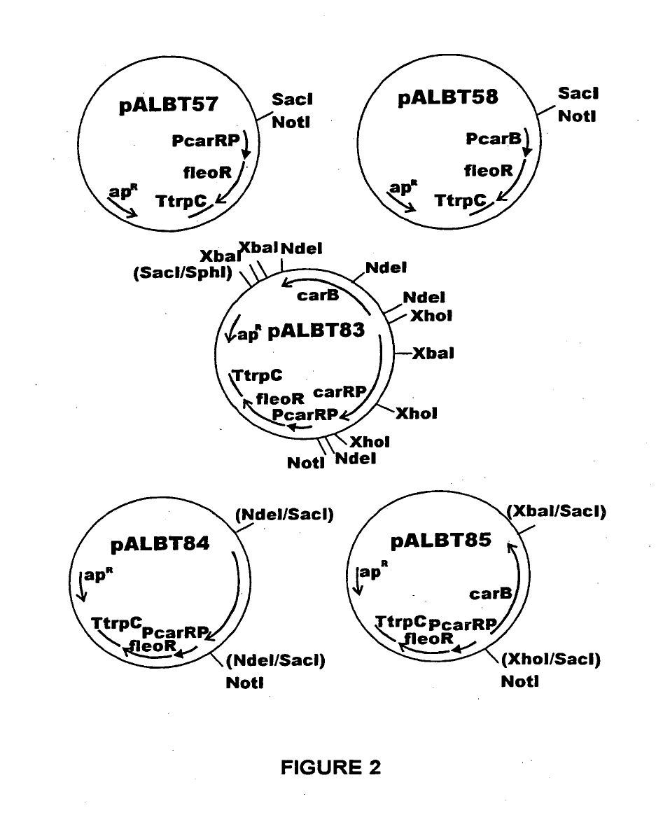

Starting from the pALBT56 plasmid, a 0.6-kb NcoI fragment was purified, with the ‘Quiaex II’ kit (Quiagen), corresponding to the bidirectional promoter of the carB and carRP genes (PcarB-PcarRP). This fragment was joined to the pALfleo8 plasmid, previously digested with the endonuclease NcoI, obtaining two different plasmids: pALBT57 (which expresses the ble^{R} gene under the PcarRP promoter) and pALBT58 (which expresses the ble^{R} gene under the PcarB promoter). The plasmids pALBT57 and pALBT58 (FIG. 2) permit the heterologous expression of the ble^{R} gene of S. hindustanus in B. trispora.

### EXAMPLE 4

Construction of Plasmids for Inserting Additional Copies of the carRP and carB Genes in B. trispora.

With the aim of inserting additional copies of the carRP and carB genes in B. trispora, the plasmids pALBT83, pALBT84 and pALBT85 were constructed (FIG. 2). In all three cases, the pALBT57 plasmid described previously was used as the starting point. The construction of pALBT83 was carried out in the following way: fALBT4 was digested with the endonuclease SphI, then its ends were blunted with the Klenow fragment of DNA polymerase I from E. coli (without addition of dNTPs for promoting the action of its exonuclease activity) and finally was digested with the endonuclease NotI. Next, a 6.9-kb fragment was purified, with the ‘Quiaex II’ kit (Quiagen), which contains the carB and carRP genes. Furthermore, the pALBT57 plasmid was digested with the endonuclease SacI, then incubated with the Klenow fragment of DNA polymerase I to obtain blunt ends (without addition of dNTPs for promoting the action of its exonuclease activity) and finally was digested with NotI. The compatible ends of the plasmid and of the insert were joined and the result of the ligation was transformed by electroporation into E. coli DH5α. The transformants were selected in LB medium (Sambrook J. et al. (1989) Molecular Cloning: A Laboratory Manual, Cold Spring Harbor Laboratory, Cold Spring Harbor, N.Y., USA) for ampicillin resistance (present in the pALBT57 vector). The desired construction was selected by means of restriction analysis, obtaining the pALBT83 plasmid (FIG. 2).

Construction of pALBT84 was carried out in the following way: pALBT9 was digested with the endonuclease NdeI and then its ends were blunted with the Klenow fragment of DNA polymerase I of E. coli. Next, a 3.5 kb fragment containing the carRP gene was purified, with the ‘Quiaex II’ kit (Quiagen). In addition, the pALBT57 plasmid was digested with the endonuclease SacI and was then incubated with the Klenow fragment of DNA polymerase I to blunt the ends (without addition of dNTPs for promoting the action of its exonuclease activity). The blunt ends of the plasmid and of the insert were joined and the result from ligation was transformed by electroporation in E. coli DH5α. The transformants were selected in LB medium (Sambrook J. et al. (1989) Molecular Cloning: A Laboratory Manual, Cold Spring Harbor Laboratory, Cold Spring Harbor, N.Y., USA) for ampicillin resistance (present in the pALBT57 vector). The desired construction was selected by restriction analysis, obtaining the pALBT84 plasmid (FIG. 2).

Construction of pALBT85 was carried out in the following way: pALBT52 was digested with the endonucleases XhoI-XbaI and then its ends were blunted with the Klenow fragment of DNA polymerase I of E. coli. Next, a 2.6-kb fragment containing the carB gene was purified, with the ‘Quiaex II’ kit (Quiagen). In addition, the pALBT57 plasmid was digested with the endonuclease SacI and was then incubated with the Klenow fragment of DNA polymerase I to blunt the ends (without addition of dNTPs for promoting the action of its exonuclease activity). The blunt ends of the plasmid and of the insert were joined and the result from ligation was transformed by electroporation in E. coli DH5α. The transformants were selected in LB medium (Sambrook J. et al. (1989) Molecular Cloning: A Laboratory Manual, Cold Spring Harbor Laboratory, Cold Spring Harbor, N.Y., USA) for ampicillin resistance (present in the pALBT57 vector). The desired construction was selected by restriction analysis, obtaining the pALBT85 plasmid (FIG. 2).

With the aim of clearly defining the invention, a total of two diagrams are presented. The abbreviations BamHI, HindIII, etc. are conventional abbreviations for restriction endonucleases. The approximate length of the DNA fragments is shown in kilobases (kb) according to the sizes determined by agarose gel electrophoresis. The diagrams do not show all of the restriction sites present. The DNA of the invention can be inserted in any suitable vector by various methods: direct ligation of cohesive ends, use of homopolymeric adhesives, by means of adapting or ligating molecules, etc.

### DETAILED DESCRIPTION OF THE DIAGRAMS

FIG. 1. Restriction map of the region of the genome of B. trispora that contains the carB and carRP genes. The arrow indicates the direction of transcription of each of them, and the rectangles indicate the presence of introns. The lines in the lower part of the diagram represent the DNA fragments of B. trispora cloned in the phage vector λ-GEM12 (fALBT) or in plasmids (pALBT). The nucleotide sequence of this DNA fragment is described in SEQ ID NO:1 and SEQ ID NO:2.

FIG. 2. Physical map of the plasmids pALBT57, pALBT58, pALBT83, pALBT84 and pALBT85. pALBT57 consists of pBluescript I KS (+) with a 1.7-kb HindIII-BamHI insert that includes the phleomycin-resistance construction PcarRP-ble^{R}-TtrpC. pALBT58 consists of pBluescript I KS (+) with a 1.7-kb HindIII-BamHI insert that includes the phleomycin-resistance construction PcarB-ble^{R}-TtrpC. pALBT83 is formed by pALBT57 with a 6.9-kb SphI-NotI insert that includes the carB and carRP genes. pALBT84 consists of pALBT57 with a 3.5-kb NdeI insert that includes the carRP gene. pALBT85 is pALBT57 with a 2.6-kb XhoI-XbaI insert that includes the carB gene.

1. A DNA compound isolated from B. trispora, defined by the restriction map shown in FIG. 1, that contains a sequence, identified as SEQ ID NO:1, which includes the carRP gene of B. trispora that codes for a polypeptide, identified as SEQ ID NO:3, showing activity lycopene cyclase/phytoene synthase and a second sequence, identified as SEQ ID NO:2, which includes the carB gene of B. trispora that codes for a polypeptide, identified as SEQ ID NO:4, showing activity phytoene dehydrogenase.

2. A polypeptide, identified as SEQ ID NO:3, showing a bifunctional enzymatic activity of lycopene cyclase/phytoene synthase, encoded by SEQ ID NO:1 contained in the DNA compound of claim 1.

3. A polypeptide, identified as SEQ ID NO:4, showing an enzymatic activity of phytoene dehydrogenase encoded by SEQ ID NO:2 of claim 1.

4. Vectors bearing the DNA compound described in claim 1.

5. Vectors according to claim 4, that express the carRP gene under the control of expression signals different from those of the gene itself.

6. Vectors according to claim 4, that express the carB gene under the control of expression signals different from those of the gene itself.

7. Vectors according to claim 5, characterized in that the expression signals are obtained from B. trispora.

8. Vectors according to claim 4, characterized in that they consist of a plasmid.

9. Plasmid according to claim 8, characterized in that it consists of pALBT9, which includes the carRP gene.

10. Plasmid according to claim 8, characterized in that it consists of pALBT52, which includes the carB gene.

11. Plasmid according to claim 8, characterized in that it consists of pALBT83, which includes the carRP and carB genes.

12. Plasmid according to claim 8, characterized in that it consists of pALBT84, which includes the carRP gene.

13. Plasmid according to claim 8, characterized in that it consists of pALBT85, which includes the carB gene.

14. Plasmid according to claim 8, characterized in that it consists of pALBT57, which includes the ble^{R} gene expressed under the control of the promoter of the carRP gene.

15. Plasmid according to claim 8, characterized in that it consists of pALBT58, which includes the ble^{R} gene expressed under the control of the promoter of the carB gen

16. Vectors according to claim 6, characterized in that the expression signals are obtained from B. trispora.

17. Vectors according to claim 5, characterized in that they consist of a plasmid.

18. Vectors according to claim 6, characterized in that they consist of a plasmid.

19. Vectors according to claim 7, characterized in that they consist of a plasmid.

20. Vectors according to claim 16, characterized in that they consist of a plasmid.


SEQUENCE LISTING (10 sequences)

SEQ ID NO: 1 - DNA, 8752 bp, Blakeslea Trispora
gggctcacttgtatcatcactgttgttaataaattgttgagaataaagcttggattgtatacgactgagccagttggagccacttgtaggccttgtgggtgatacaacagcaggctttgatacagtggtatttatttctggcctaaacttcaatatactttttggactgttcatgccttgatataacaaaataaaataaaaagggaccttgtacactgaaagccaaatgaataaaataaaaaataaaaaataaaaagttatttgcagaaacccaaaaagcccgaaatttcttctaaaagaaagaaaataaaaaaaaagagagacttacttttaggcgatcaacagtcttttttttttgcttgagcttcttttaagctttacatttaggcacttttataattgctgtattgaatatgacatctatcttttttaacttgaacgatttgtagctgttttctcttgacaatgttaattttaaagcatgattttttttctgaaaaaataaagaccgcttacataatacaaaagtcagaattgtatggctattttatcctgtctcttcttttcaggaactcaaccatctccttgtctttattgtctgagaatgctttatctgactcttattaatcctttgagaaaccaaaaatggggtggattgagacttggactcgctttgtgcatttgctccttctgttcgatcattattgagcaatgtcatacaaacattcaatgacaagcaacatcaaactaggagacagattaagcaagcagatgatttataaagggctactctgctcattgatttacatgctttagcatcatgaattgtgttgcttccattccaaaatagatgtctttttctctttccacttgcctctttctttttttggctattcagcataatgcaagagtagctattcctcaacccatcagtaataagtgaatggcacggtagcattgtgtttgtatgttttctatctgcgttttgattttcgaatctcatctttaggatagtatcttgagattgtattgattttgcctgatacacttttgtttttgtgaatatgagcttcagggaagtgatctgtttaagtacaattgaaataaagtgatagtctttgaggtttggtatatggctttctaaagcatggacagagccatgacaaaaaaaagggcggtataaagcctcagataactccctctcttgcactctgacaaatgtagtacaggcttattgccaatggaatgactcaatagatggctaaatgtgaagaaaaacgtattggaaaagcctacaaaactttccttaataaacatcatggccatcttttttgaaagatttcaaagactaggttaaaaataccattactatcatcttgcttctagtcctctgactatttctttgttttcaacaacacgtgtaggaaggacaagaaagatgtaagcgacagaatagcttagttttactatactctacttcttttttttttgtagttttccatcttccttgctttgaagatagacaattttactcacacaatttctttttcgttaaagctgatagcttgaatatcatcatatcaaacagaagacatatatcgctaaatagtacttgctataacagagaaagcgatccatgtgcaactctgatgcttatcgccgtagacgactttccttaaaaaaaactgacaaacaccgtagaatgtgacacatacacacaacaatactgttgctcaaaactttaactaatagttaaagtacaatgaaaatatattgcagtctagaatgataacaattttgctttaacgatgtggtgcagtcttaattcaaagctcaaagaaaagaaaaagaatttgctagctatcatgagctaaatctctgtttttcttgaaacaatcttacaaagaaaagctatattgcttgacaaagggcaaatccaccgagattcttttactgcatgcccgaataaaaagggagaggaagaggagtatgttacacttgaatgtatttttgaggaagcatccgcattaatttggtgttataaacactgagtaatcatgttatttgaagactgaacctttaccaaaaaggtcttgtagatcgcttgttgcaattgagatgggttaaaaataagtctaaagttaagataagcacaatgaaaagaacgtttttattttctatcaggcaaagtaaaaccacttttctagatgtggcaataagcaatcaagccaaagggagaaaaaagcttatcaaagctatggctttcaagagaatagtaatttaggtactacacaaagccactgtttatgcttctttgcaatatcaacaaagagacattgtgtctgttgaaatgttttgtttgacatgtttaatcagatcaagtgaggatgctttactctttggtttagtaaaagaaacacaccagcaactccggtgaatgttatgattatgacgtttcaaacgaaaaatctctattttcgttttaaggttagtccttttagaataccgtttttttttttaccatttcattgtcttgaaaaccctcccaagctaatgattattttctttttttacgcaagactcgtatcactcacctaccttacagacgtgttttgcttttttggataatgctgtgcttgatctatgtatggctcctttgccttatttttaaaaagaaatgtttgctaggattgatttttaatggttactctcaatcaaataccacatttagtagaaacaaaatttgtgcatatcataataaaactaaattcgattattttttctaaaatcaggataatttgtttttccaatatttgtttgtagaattgtctgtcctaccaaacaattcagttttctatttgcgtcgagtcatttattttgggtttctttgtttgagctgattctgatacacatgtgaattgtctttttagacactattctagaattcattccattcgaaaggatcaacatacaccaatttaatgacgtgctagataatggatacaaatatacgcacaaaaaaagaaagaattctatgatcaaagagaacgcagacacagagtgatacatttaaatggttaagttcatatgatgttaaaatggtagctttattattgaactaaatgcgaatatcgttgctgttttgtccttggaaaacgttaggtaaaagttggttaatgaaagaagcaggagttgtagtatcatctcttgggaagaaatagaaaaagaggaaagtaacaaagtaacaagcaagacaataatagatccaatggctttcggtcttacgagtttgttcaggagcatacttcttttggctatcttgtaactttcttggtaagggattctggccaaagcttttacagacttggtcggaagtaagcttacttccagcaagaacgataggaacaccagtacctggatgtgtactacaaagaaaagagaaatgagtacgtgcgttattaaaaaaaagaaaaaaagagggcaaaagtattacctagctccgacaaagaaaagattatcataacggtttgtggaatccttggtactaggtctgaaccagagaacttggaacacatcatgagaaagaccaagaatagaacctctccaaaggttaaacttgctttgccaaacactaggatcattcacttcttcatgttcaatcaaattagcaaagttgtttactcccaaacgacgttcgataacttccagaaccatcttgcgtgcacggtttaccaactcaggataattttcttcagcactgtttcctgtcttactcttcatatggccaattggaaccaacacaataatggagtccttgttgggaggtgcggcagattcatcaattcgagatggaacgttgacatagaatgaagcttcagagggcaaaccgaagtcgttgaaaatctcatcaaaactttccttgtaggcttcagccaagaagatattgtgtacgtctaattgaggcacctttgttgacatggaccaataaaacgaaatagatgatgaagtgagtttctttgaggctaatgtcttctttgtccaattgcaaggaggtaacagatggtgataagcataaacaagatccgcattacatacgactgcatcggcttcaatgacttctccgctttccaaagtgacaccggttacacgcttgtctttatcgacagtgttaattttagcaacaggcgattgatatctgaattcagcaccgtactttttggaggcgatagactcaagcttctgaacaaccatgttgaaaccaccacgaggataccagataccttcagcaaactcggtgtattgtaacaaactgtaaactgctggagcatcataaggcgacatactatattccaaaaatagaaaatagaacaatgaatatcaaaattcctttcacttgccctttttcacatttctcttttcccacccccgaccggtctcactcatttttttttcatcccacaccacgcgttgtatgtgtacttaccccatatacattgtttgaaaagtaaaagccatacgcattttcttggtttggaaatatttactggctcggtcatagatcttaccaaacaagtgcaagcgaaagatttcaggcacatactgaagacgaatcaaatcccaaatggtttcaaagttgcgcttgatagcaataaatgtaccttgttcataatggacatgtgtttccttcatgaaatccaagaatctaccaaatccaaggggaccctcaatacggtccaattcgcccttcatcttggttaaatcggaagagagttgtacggcatcaccgtcgtcaaaatgaaccttatagttattgtcacagcgaagcaaatccaaatgatcaccaatacgttcatccaaatcagcaaatgcatcttcaaaaagcttaggcatcaaatagagtgagggaccctgatcaaagcgatgaccatcgtgatgaatgaatgaacaacggccaccggaaaagtcgttcttttcaacaacagtaactcgaaaaccttcacgagcaagacgagcagcagtagcagttccgccaataccggcaccaatgacaacaatatgcttcttttgatcagacatgagattaaaatagataaggaaaagaaagtgaaaagaaattcggaagcatggcacattcttctttttataaatacatgcctgactttctttttccatcgatatgatatatgcatatgatagatatacaagcaatcttcttcaaggagtttgaaattttgtcctccaggagcaaaaaaaagtttttttttatacatgtttgtacacaagaatagttaccaatttgctttggtcttacgtgctgcaagtttatatcgttttcaatttctttgtctttacattttctttgtcctttatctttcctcatttagtctttgggagaattaggaaaagggagcggaaaggtaagaaatgcttgcgtattttactaattcggcaaacatccaatttggcaaacagcagcctgtgcaacgctctcgagatgacagtatctttgattacactctaaatctcgatgacccgaccaaaaagagcgaacaaagaaataatcttgtgcattcgaatatgatggaagattttttcccccttattctaaatgttgacatagcgtgtatgttatataaacaaaaagaaattgtacaaactttcttttcttctctttttattttatctctatgtcaatactcacttatctggaatttcatctctactatacactacctgtccttgcggcattgtgttggctgctaaagccgtttcactcacagcaagacaataagtataaatttttaatgttgatggccgcctctaccgcatcgattgacaattatatcgtttatcatcgcgcttggtggtactgtcctacttgtgttgtggctgtcattggctatgtacctctagaagaatacatgttcatcatcatgactttaatgactgtcgcgttctcaaactttgttatgtggcacttgcatactttctttattagacccaacacttcttggaagacactattagtacgccttgtgcctgtttcagctttattggcaatctatcatgcttgggtatgcaaaataaacaaacactaaaaaaaataattattttactcatttttctttttttagcacttgacactgccaaataaatcattttatggttcatgcatcctttggtatgcttgtcctgtgttgattctttggctgggtgctggtgaatatatcttgcgtcgacctgtggctgtccttttgtctattgttatccctagtgtatacctatgttgggctgatatcgtcgctattagtgctggcacatggcatatttctcttagaacaagcactggcaaaatggtagtacccgatttacctgtagaagaatgcctgttttttactttgatcaacacagtcttggtttttgctacctgtgctatagaccgcgctcaggccatcctccatctgtacaaatcatctgttcaaaatcaaaaccctaaacaagccatttcccttttccagcatgtcaaagagctagcatgggccttctgtcttcctgaccaaatgctcaacaatgaattgtttgatgatcttactatcagctgggatattttacgtaaagcctcaaagtcattctatactgcatctgccgtttttccaagttatgtacgtcaagacttgggtgttctctatgctttctgcagagctaccgatgacctgtgcgatgatgaatccaaatctgttcaagaaagaagagaccaattagatcttactcgacaatttgttcgtgatctctttagccaaaagaccagtgcgcctattgtgattgattgggaattgtatcaaaaccaacttcctgcttcttgtatatcagcctttagagcctttactcgccttcgccatgtccttgaagtagaccctgtagaagaactattagatggttacaaatgggatcttgagcgtcgtcctatccttgatgaacaagacttggaggcatactctgcttgtgtggccagtagtgtgggtgaaatgtgcacacgtgtgattcttgctcaagaccaaaaggaaaatgatgcttggataattgaccgtgcacgtgagatggggctggtgctacaatacgttaacattgctcgagacattgtgactgatagcgagactctgggtcgatgttatctgcctcaacaatggcttagaaaagaagaaacagaacaaatacagcaaggcaacgcccgtagcctaggtgatcaaagactgttgggcttgtctctgaagcttgtaggaaaggcagacgctatcatggtgagagctaagaagggcattgacaagttgccggcaaactgtcaaggcggtgtacgagctgcttgccaagtatatgctgcaattggatctgtactcaagcagcagaagacaacatatcctacaagagctcatctaaaaggaagcgaacgtgccaagattgctctgttgagtgtatacaacctctatcaatctgaagacaagcctgtggctctccgtcaagctagaaagattaagagtttttttgttgattagtgaatttttgttttatttatgtctgatagttcaataaagagacaacacatacaatataaaatcattgtctttaaatgttaatttagtagagtgtaaagcctgcattttttttgtacgcataaacaatgagttcaccccgcttctggtttttaaataattatgtcaaactagggaaaattcttttttttctcttcgttctttttttggcttgttgtggagtcacaggcttgtcttcagattgatagaggttgtatacactcaacagagcaatcttggcacgttcgcttccttttagatgagctcttgtaggatatgttgtcttctgctgcttgagtacagatccaattgcagcatatacttggcaagcagctcgtacaccgccttgacagtttgccggcaacttgtcaatgcccttcttagctctcaccatgatagcgtctgcctttcctacaagcttcagagacaagcccaacagtctttgatcacctaggctacgggcgttgccttgctgtatttgttctgtttcttcttttctaagccattgttgaggcagataacatcgacccaacatcctcgagccatactacagcataaaaggatacgttttctttaacagaaatttacccttttgttatcagcacatacaaaaaaaaagaaatttaagatgagtaggacttccattctctcaaaaattttattcaatccataaatgaattatttttggacaaaaaagaaagattatgcctgattttctctattttttttttttttacaactccaccaatactttctagagacacatttgagcgatgtgacagtcggactcgagaagtacaagaaggtacagaaatagcagctgagcgtatgattgggtcccaaggttcttctcgactttctctcttaccctggaacagagagaaaaaaaaaatatttcgtcttttttggataatattataaaaaagggaatttagtaaagaaaacggttgtttccttttctttttttttttccttctccactacatgaataaacatcgccacccaaatttaccttccatatctactctacttactggaccaccttctccgccacctcccattattgtgatagatgaggaacattctcccagttgttcacctaacaaataccacttgtctcctgtcttatcacctattgattcatatgcttcatcaccaaactcgag

SEQ ID NO: 2 - DNA, 8783 bp, Blakeslea Trispora
ctcgagtttggtgatgaagcatatgaatcaataggtgataagacaggagacaagtggtatttgttaggtgaacaactgggagaatgttcctcatctatcacaataatgggaggtggcggagaaggtggtccagtaagtagagtagatatggaaggtaaatttgggtggcgatgtttattcatgtagtggagaaggaaaaaaaaaaagaaaaggaaacaaccgttttctttactaaattcccttttttataatattatccaaaaaagacgaaatattttttttttctctctgttccagggtaagagagaaagtcgagaagaaccttgggacccaatcatacgctcagctgctatttctgtaccttcttgtacttctcgagtccgactgtcacatcgctcaaatgtgtctctagaaagtattggtggagttgtaaaaaaaaaaaaaatagagaaaatcaggcataatctttcttttttgtccaaaaataattcatttatggattgaataaaatttttgagagaatggaagtcctactcatcttaaatttctttttttttgtatgtgctgataacaaaagggtaaatttctgttaaagaaaacgtatccttttatgctgtagtatggctcgaggatgttgggtcgatgttatctgcctcaacaatggcttagaaaagaagaaacagaacaaatacagcaaggcaacgcccgtagcctaggtgatcaaagactgttgggcttgtctctgaagcttgtaggaaaggcagacgctatcatggtgagagctaagaagggcattgacaagttgccggcaaactgtcaaggcggtgtacgagctgcttgccaagtatatgctgcaattggatctgtactcaagcagcagaagacaacatatcctacaagagctcatctaaaaggaagcgaacgtgccaagattgctctgttgagtgtatacaacctctatcaatctgaagacaagcctgtgactccacaacaagccaaaaaaagaacgaagagaaaaaaaagaattttccctagtttgacataattatttaaaaaccagaagcggggtgaactcattgtttatgcgtacaaaaaaaatgcaggctttacactctactaaattaacatttaaagacaatgattttatattgtatgtgttgtctctttattgaactatcagacataaataaaacaaaaattcactaatcaacaaaaaaactcttaatctttctagcttgacggagagccacaggcttgtcttcagattgatagaggttgtatacactcaacagagcaatcttggcacgttcgcttccttttagatgagctcttgtaggatatgttgtcttctgctgcttgagtacagatccaattgcagcatatacttggcaagcagctcgtacaccgccttgacagtttgccggcaacttgtcaatgcccttcttagctctcaccatgatagcgtctgcctttcctacaagcttcagagacaagcccaacagtctttgatcacctaggctacgggcgttgccttgctgtatttgttctgtttcttcttttctaagccattgttgaggcagataacatcgacccagagtctcgctatcagtcacaatgtctcgagcaatgttaacgtattgtagcaccagccccatctcacgtgcacggtcaattatccaagcatcattttccttttggtcttgagcaagaatcacacgtgtgcacatttcacccacactactggccacacaagcagagtatgcctccaagtcttgttcatcaaggataggacgacgctcaagatcccatttgtaaccatctaatagttcttctacagggtctacttcaaggacatggcgaaggcgagtaaaggctctaaaggctgatatacaagaagcaggaagttggttttgatacaattcccaatcaatcacaataggcgcactggtcttttggctaaagagatcacgaacaaattgtcgagtaagatctaattggtctcttctttcttgaacagatttggattcatcatcgcacaggtcatcggtagctctgcagaaagcatagagaacacccaagtcttgacgtacataacttggaaaaacggcagatgcagtatagaatgactttgaggctttacgtaaaatatcccagctgatagtaagatcatcaaacaattcattgttgagcatttggtcaggaagacagaaggcccatgctagctctttgacatgctggaaaagggaaatggcttgtttagggttttgattttgaacagatgatttgtacagatggaggatggcctgagcgcggtctatagcacaggtagcaaaaaccaagactgtgttgatcaaagtaaaaaacaggcattcttctacaggtaaatcgggtactaccattttgccagtgcttgttctaagagaaatatgccatgtgccagcactaatagcgacgatatcagcccaacataggtatacactagggataacaatagacaaaaggacagccacaggtcgacgcaagatatattcaccagcacccagccaaagaatagccaacacaggacaagcataccaaaggatgcatgaaccataaaatgaagatttatttggcagtgtcaagtgctaaaaaaagaaaaatgagtaaaataattatcgctattatttttttttagtgtttgtttattttgcatacccaagcatgataagtgattgccaataaagctgaaacaggcacaaggcgtactaatagtgtttgcttccaagaagtgttgggtctaataaagaaagtatgcaagtgccaacgcataacaaagtttgagaacgcgacagtcattaaagtcatgatgataaagaacatgtattcttctagaggtacatagccaatgacagccacaacacaagtaggacagtaccaccaagcgcgatgataaacgatataattgtcccaaatcgatgcggtagaggcggccatcaacattaaaaatttatacttgagattgtcttgctgtgagtgaaacggctttagcagccaacacaatgccgcaaggacaggtagtgtatagtagagatgaaattccagataagtgagtattgacatagagataaaataaaaagagaagaaaagaaagtttgtacaatttctttttgtttatataacatacacgctatgtcaacatttagaataagggggaaaaaatcttccatcatattcgaatgcacaagattatttctttgttcgctctttttggtcgggtcatcgagatttagagtgtaatcaaagatactgtcatctcgagagcgttgcacaggctgctgtttgccaaattggatgtttgccgaattagtaaaatacgcaagcatttcttacctttccgctcccttttcctaattctcccaaagactaaatgaggaaagataaaggacaaagaaaatgtaaagacaaagaaattgaaaacgatataaacttgcagcacgtaagaccaaagcaaattggtaactattcttgtgtacaaacatgtataaaaaaaaacttttttttgctcctggaggacaaaatttcaaactccttgaagaagattgcttgtatatctatcatatgcatatatcatatcgatggaaaaagaaagtcaggcatgtatttataaaaagaagaatgtgccatgcttccgaatttcttttcactttcttttccttatctattttaatctcatgtctgatcaaaagaagcatattgttgtcattggtgccggtattggcggagctactgctgctcgtcttgctcgtgaaggttttcgagttactgttgttgaaaagaacgacttttccggtggccgttgttcattcattcatcacgatggtcatcgctttgatcagggtccctcactctatttgatgcctaagctttttgaagatgcatttgctgatttggatgaacgtattggtgatcatttggatttgcttcgctgtgacaataactataaggttcattttgacgacggtgatgccgtacaactctcttccgatttaaccaagatgaagggcgaattggaccgtattgagggtccccttggatttggtagattcttggatttcatgaaggaaacacatgtccattatgaacaaggtacatttattgctatcaagcgcaactttgaaaccatttgggatttgattcgtcttcagtatgtgcctgaaatctttcgcttgcacttgtttggtaagatctatgaccgagccagtaaatatttccaaaccaagaaaatgcgtatggcttttacttttcaaacaatgtatatggggtaagtacacatacaacgcgtggtgtgggatgaaaaaaaaatgagtgagaccggtcgggggtgggaaaagagaaatgtgaaaaagggcaagtgaaaggaattttgatattcattgttctattttctatttttggaatatagtatgtcgccttatgatgctccagcagtttacagtttgttacaatacaccgagtttgctgaaggtatctggtatcctcgtggtggtttcaacatggttgttcagaagcttgagtctatcgcctccaaaaagtacggtgctgaattcagatatcaatcgcctgttgctaaaattaacactgtcgataaagacaagcgtgtaaccggtgtcactttggaaagcggagaagtcattgaagccgatgcagtcgtatgtaatgcggatcttgtttatgcttatcaccatctgttacctccttgcaattggacaaagaagacattagcctcaaagaaactcacttcatcatctatttcgttttattggtccatgtcaacaaaggtgcctcaattagacgtacacaatatcttcttggctgaagcctacaaggaaagttttgatgagattttcaacgacttcggtttgccctctgaagcttcattctatgtcaacgttccatctcgaattgatgaatctgccgcacctcccaacaaggactccattattgtgttggttccaattggccatatgaagagtaagacaggaaacagtgctgaagaaaattatcctgagttggtaaaccgtgcacgcaagatggttctggaagttatcgaacgtcgtttgggagtaaacaactttgctaatttgattgaacatgaagaagtgaatgatcctagtgtttggcaaagcaagtttaacctttggagaggttctattcttggtctttctcatgatgtgttccaagttctctggttcagacctagtaccaaggattccacaaaccgttatgataatcttttctttgtcggagctaggtaatacttttgccctctttttttcttttttttaataacgcacgtactcatttctcttttctttgtagtacacatccaggtactggtgttcctatcgttcttgctggaagtaagcttacttccgaccaagtctgtaaaagctttggccagaatcccttaccaagaaagttacaagatagccaaaagaagtatgctcctgaacaaactcgtaagaccgaaagccattggatctattattgtcttgcttgttactttgttactttcctctttttctatttcttcccaagagatgatactacaactcctgcttctttcattaaccaacttttacctaacgttttccaaggacaaaacagcaacgatattcgcatttagttcaataataaagctaccattttaacatcatatgaacttaaccatttaaatgtatcactctgtgtctgcgttctctttgatcatagaattctttctttttttgtgcgtatatttgtatccattatctagcacgtcattaaattggtgtatgttgatcctttcgaatggaatgaattctagaatagtgtctaaaaagacaattcacatgtgtatcagaatcagctcaaacaaagaaacccaaaataaatgactcgacgcaaatagaaaactgaattgtttggtaggacagacaattctacaaacaaatattggaaaaacaaattatcctgattttagaaaaaataatcgaatttagttttattatgatatgcacaaattttgtttctactaaatgtggtatttgattgagagtaaccattaaaaatcaatcctagcaaacatttctttttaaaaataaggcaaaggagccatacatagatcaagcacagcattatccaaaaaagcaaaacacgtctgtaaggtaggtgagtgatacgagtcttgcgtaaaaaaagaaaataatcattagcttgggagggttttcaagacaatgaaatggtaaaaaaaaaaacggtattctaaaaggactaaccttaaaacgaaaatagagatttttcgtttgaaacgtcataatcataacattcaccggagttgctggtgtgtttcttttactaaaccaaagagtaaagcatcctcacttgatctgattaaacatgtcaaacaaaacatttcaacagacacaatgtctctttgttgatattgcaaagaagcataaacagtggctttgtgtagtacctaaattactattctcttgaaagccatagctttgataagcttttttctccctttggcttgattgcttattgccacatctagaaaagtggttttactttgcctgatagaaaataaaaacgttcttttcattgtgcttatcttaactttagacttatttttaacccatctcaattgcaacaagcgatctacaagacctttttggtaaaggttcagtcttcaaataacatgattactcagtgtttataacaccaaattaatgcggatgcttcctcaaaaatacattcaagtgtaacatactcctcttcctctccctttttattcgggcatgcagtaaaagaatctcggtggatttgccctttgtcaagcaatatagcttttctttgtaagattgtttcaagaaaaacagagatttagctcatgatagctagcaaattctttttcttttctttgagctttgaattaagactgcaccacatcgttaaagcaaaattgttatcattctagactgcaatatattttcattgtactttaactattagttaaagttttgagcaacagtattgttgtgtgtatgtgtcacattctacggtgtttgtcagttttttttaaggaaagtcgtctacggcgataagcatcagagttgcacatggatcgctttctctgttatagcaagtactatttagcgatatatgtcttctgtttgatatgatgatattcaagctatcagctttaacgaaaaagaaattgtgtgagtaaaattgtctatcttcaaagcaaggaagatggaaaactacaaaaaaaaaagaagtagagtatagtaaaactaagctattctgtcgcttacatctttcttgtccttcctacacgtgttgttgaaaacaaagaaatagtcagaggactagaagcaagatgatagtaatggtatttttaacctagtctttgaaatctttcaaaaaagatggccatgatgtttattaaggaaagttttgtaggcttttccaatacgtttttcttcacatttagccatctattgagtcattccattggcaataagcctgtactacatttgtcagagtgcaagagagggagttatctgaggctttataccgcccttttttttgtcatggctctgtccatgctttagaaagccatataccaaacctcaaagactatcactttatttcaattgtacttaaacagatcacttccctgaagctcatattcacaaaaacaaaagtgtatcaggcaaaatcaatacaatctcaagatactatcctaaagatgagattcgaaaatcaaaacgcagatagaaaacatacaaacacaatgctaccgtgccattcacttattactgatgggttgaggaatagctactcttgcattatgctgaatagccaaaaaaagaaagaggcaagtggaaagagaaaaagacatctattttggaatggaagcaacacaattcatgatgctaaagcatgtaaatcaatgagcagagtagccctttataaatcatctgcttgcttaatctgtctcctagtttgatgttgcttgtcattgaatgtttgtatgacattgctcaataatgatcgaacagaaggagcaaatgcacaaagcgagtccaagtctcaatccaccccatttttggtttctcaaaggattaataagagtcagataaagcattctcagacaataaagacaaggagatggttgagttcctgaaaagaagagacaggataaaatagccatacaattctgacttttgtattatgtaagcggtctttattttttcagaaaaaaaatcatgctttaaaattaacattgtcaagagaaaacagctacaaatcgttcaagttaaaaaagatagatgtcatattcaatacagcaattataaaagtgcctaaatgtaaagcttaaaagaagctcaagcaaaaaaaaaagactgttgatcgcctaaaagtaagtctctctttttttttattttctttcttttagaagaaatttcgggctttttgggtttctgcaaataactttttattttttattttttattttattcatttggctttcagtgtacaaggtccctttttattttattttgttatatcaaggcatgaacagtccaaaaagtatattgaagtttaggccagaaataaataccactgtatcaaagcctgctgttgtatcacccacaaggcctacaagtggctccaactggctcagtcgtatacaatccaagctttattctcaacaatttattaacaacagtgatgatacaagtgagccc

SEQ ID NO: 3 - protein, 608 aa, Blakeslea Trispora
MSILTYLEFHLYYTLPVLAALCWLLKPFHSQQDNLKYKFLMLMAASTASIWDNYIVYHRAWWYCPTCVVAVIGYVPLEEYMFFIIMTLMTVAFSNFVMRWHLHTFFIRPNTSWKQTLLVRLVPVSALLAITYHAWHLTLPNKSSFYGSCILWYACPVLAILWLGAGEYILRRPVAVLLSIVIPSVYLCWADIVAISAGTWHISLRTSTGKMVVPDLPVEECLFFTLINTVLVFATCAIDRAQAILHLYKSSVQNQNPKQAISLFQHVKELAWAFCLPDQMLNNELFDDLTISWDILRKASKSFYTASAVFPSYVRQDLGVLYAFCRATDDLCDDESKSVQERRDQLDLTRQFVRDLFSQKTSAPIVIDWELYQNQLPASCISAFRAFTRLRHVLEVDPVEELLDGYKWDLERRPILDEQDLEAYSACVASSVGEMCTRVILAQDQKENDAWIIDRAREMGLVLQYVNIARDIVTDSETLGRCYLPQQWLRKEETEQIQQGNARSLGDQRLLGLSLKLVGKADAIMVRAKKGIDKLPANCQGGVRAACQVYAAIGSVLKQQKTTYPTRAHLKGSERAKIALLSVYNLYQSEDKPVALRQARKIKSFFVD

SEQ ID NO: 4 - protein, 582 aa, Artificial
  Deduced amino acid sequence encoded by the carB of B. Trispora.
MSDQKKHIVVIGAGIGGTATAARLAREGFRVTVVEKNDFSGGRCSFIHHDGHRFDQGPSLYLMPKLFEDAFADLDERIGDHLDLLRCDNNYKVHFDDGDAVQLSSDLTKMKGELDRIEGPLGFGRFLDFMKETHVHYEQGTFIAIKRNFETIWDLIRLQYVPEIFRLHLFGKIYDRASKYFQTKKMRMAFTFQTMYMGMSPYDAPAVYSLLQYTEFAEGIWYPRGGFNMVVQKLESIASKKYGAEFRYQSPVAKINTVDKDKRVTGVTLESGEVIEADAVVCNADLVYAYHHLLPPCNWTKKTLASKKLTSSSISFYWSMSTKVPQLDVHNIFLAEAYKESFDEIFNDFGLPSEASFYVNVPSRIDESAAPPNKDSIIVLVPIGHMKSKTGNSAEENYPELVNRARKMVLEVIERRLGVNNFANLIEHEEVNDPSVWQSKFNLWRGSILGLSHDVFQVLWFRPSTKDSTNRYDNLFFVGASTHPGTGVPIVLAGSKLTSDQVCKSFGQNPLPRKLQDSQKKYAPEQTRKTESHWIYYCLACYFVTFLFFYFFPRDDTTTPASFINQLLPNVFQGQNSNDIRI

SEQ ID NO: 5 - DNA, 20 bp, Artificial
  PCR Primer
cgcgccgactgccattgact

SEQ ID NO: 6 - DNA, 19 bp, Artificial
  PCR Primer
cacgcacgccgccttgaca

SEQ ID NO: 7 - DNA, 28 bp, Artificial
  oligonueleotide for inserting the cutting site NcoI in the carB gen
ctattttaatcccatggctgatcaaaag

SEQ ID NO: 8 - DNA, 28 bp, Artificial
  oligonueleotide for inserting the cutting site NcoI in the carB gen
cttttgatcagccatgggattaaaatag

SEQ ID NO: 9 - DNA, 31 bp, Artificial
  oligonueleotide for inserting the cutting site NcoI in the carB gen
ctttttattttatctccatggcaatactcac

SEQ ID NO: 10 - DNA, 31 bp, Artificial
  oligonueleotide for inserting the cutting site NcoI in the carB gen
gtgagtattgccatggagataaaataaaaag